(19) United States
(12) Patent Application Publication
(10) Pub. No.: US 2017/0059555 A1
(43) Pub. Date: Mar. 2, 2017

(54) SAMPLE PLATFORM AND METHODS OF USE

(71) Applicant: Los Alamos National Security, LLC, Los Alamos, NM (US)

(72) Inventors: Rashi Iyer, Los Alamos, NM (US); Piyush Bajaj, Norwich, CT (US); Jen-Huang Huang, Los Alamos, NM (US)

(73) Assignee: Los Alamos National Security, LLC, Los Alamos, NM (US)

(21) Appl. No.: 15/252,082

(22) Filed: Aug. 30, 2016

Related U.S. Application Data
(60) Provisional application No. 62/212,254, filed on Aug. 31, 2015.

(51) Int. Cl. G01N 33/50; C12M 1/34; C12M 1/32; C12M 1/42; C12M 3/00; C12M 1/12

(52) U.S. Cl. CPC G01N 33/5005; C12M 23/42; C12M 25/02; C12M 23/12; C12M 35/00; C12M 41/46; G01N 2800/40

(57) ABSTRACT

Disclosed herein is a platform for holding a biological sample with a movable radiation source that can be used to expose the sample to controlled dosages of radiation in a targeted manner. In some embodiments, the platform includes a support structure and a cartridge for holding a sample. In some embodiments, the support structure includes a support for holding the sample, which may be in a well in the support structure. A radiation source can be adjusted relative to the sample (for example, the distance between the sample and the radiation source is controlled) in order to administer a particular dose of radiation to the sample.

### CROSS REFERENCE TO RELATED APPLICATION

This application claims the benefit of U.S. Provisional Application No. 62/212,254, filed Aug. 31, 2015, which is incorporated herein by reference in its entirety.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

This disclosure relates to platforms for holding a sample, particularly for culturing one or more cell types and/or for exposing biological samples to controlled amounts of radiation.

### BACKGROUND

Radiotherapy or radiosurgery, even though highly targeted, exposes multiple types of cells in the tissue being treated to radiation. For example, radiosurgery for AVM exposes vessel endothelial cells, fibroblasts, and/or neuronal cells to radiation. Thus, in vitro systems for studying the effect of radiation on multiple interacting cell types could improve AVM treatment, or other radiosurgery therapies (for example, tumor ablation). Furthermore, in vitro systems can be used for studying multiple cell types (such as interactions between two or more cell types, or effects of compounds on two or more interacting cell types).

### SUMMARY

Disclosed herein is a platform for holding a sample (such as a biological sample). Platforms of the disclosure include a support structure including at least one well for holding a sample. In particular embodiments, the platforms can be utilized to culture one or more cell types (such as two, three, or more cell types).

In some embodiments, the sample platform includes a support structure comprising a plurality of stacked substrates that include or form at least one well and one or more fluid inlets and/or outlets fluidly connected to the at least one well; and a removable sample cartridge including a sample support. In some examples, the sample support is a flat surface (such as a membrane) having a top surface and a bottom surface. In particular examples, the sample cartridge can be inserted in or removed from the well of the support structure. In further examples, the support structure further includes a chamber, for example, under and fluidly connected to the well, for example that can hold a fluid (such as culture medium). In particular examples, the sample cartridge can be inserted in the well of the support structure with either the top surface of the sample support facing the chamber or with the bottom surface of the sample support facing the chamber.

In some embodiments, the platform or a platform assembly further includes a movable radiation source that can be used to expose the sample to controlled dosages of radiation in a targeted manner. In some examples, the biological sample includes one or more cell types. Thus, in some embodiments, the platform includes a support structure for holding a sample (for example one or more types of cells) and a radiation source that is either placed at a fixed distance from the sample (e.g., based on the height of the support structure) or that is adjustable with respect to the distance from the sample.

In some embodiments, the support structure includes a support for holding the sample, which may be in a well in the support structure.

In certain embodiments, the radiation source can be adjusted relative to the sample (for example, the distance between the sample and the radiation source is controlled) in order to administer a particular dose of radiation to the sample. In addition to controlled radiation exposure, the platform disclosed herein minimizes the risk of radioactivity from the radiation source contaminating the sample, for example, due to inadvertent contact between the sample and the radiation source.

Also disclosed are methods including placing a sample on the sample support in the sample cartridge of the sample platform, inserting the sample cartridge in the well of the support structure, and incubating the sample for a selected period of time. The sample cartridge is then removed from the well of the support structure and one or more characteristics of the sample are evaluated. In some examples, the methods further include irradiating the sample prior to removing the sample cartridge and evaluating one or more characteristics of the sample.

The foregoing and other features of the disclosure will become more apparent from the following detailed description, which proceeds with reference to the accompanying figures.

### BRIEF DESCRIPTION OF THE DRAWINGS

FIGS. 1A-1D are a series of panels illustrating aspects of a sample platform disclosed herein (also referred to herein as “platform 1”). FIG. 1A is a schematic diagram of a cross-section of an exemplary well of the platform, showing a sample support (such as a Transwell® membrane) suspended in a culture dish with an optional wire or filament radiation source passing above the support. In this embodiment, cells are plated on both sides of the support (cell types 1 and 2) and also on the bottom of the culture dish (cell type 3). The cells may be the same cell type, or one or more different cell types. FIG. 1B is perspective view of an embodiment of platform 1. FIG. 1C is a schematic diagram of a top view of an embodiment of platform 1. FIG. 1D is a schematic of a cross-sectional view of the sample vessel (left) showing the wire radiation source and the sample support. The right panel is an expanded view of the boxed area showing that the radiation source is about 100 μm above the support in this embodiment.

FIGS. 2A-2D are schematic drawings of an exemplary embodiment of a sample platform (referred to herein as “platform 2”). FIG. 2A is a perspective view of an embodiment of platform 2 with the sample cartridge shown above the support structure. FIG. 2B is also a perspective view of an embodiment of platform 2 illustrating that the sample cartridge can be inverted before placement in the support structure. FIG. 2C is perspective view of an embodiment of platform 2 with the sample cartridge inserted in the well of the support structure. FIG. 2D is a perspective view of a cross-section of the well of the support structure, with the sample cartridge inserted.

FIGS. 3A and 3B are a series of digital images of cells plated with or without a wire radiation source. FIG. 3A is a series of five digital images showing C2C12 cells cultured on membranes with the indicated coatings. FIG. 3B is a series of three digital images showing C2C12 plated with a platinum wire directly on the membrane (left), with a platinum wire 100 μm above the membrane (middle), or without a wire (right).

FIGS. 4A-4D is a series of digital images showing culture of endothelial and neuronal cells on opposite sides of a membrane with a platinum wire. FIGS. 4A and 4B show neuron cells on the bottom side of membrane (FIG. 4A) and after 3 days in the device (FIG. 4B). FIGS. 4C and 4D shows endothelial cells (human microvascular cells) on the top side of membrane with the wire was set above the membrane about 100 μm (FIG. 4C) and after 3 days of cell culture in the device (FIG. 4D).

FIGS. 5A-5D is a series of images showing culture of cells on the top (FIG. 5A) and bottom (FIG. 5B) of the membrane in an embodiment of platform 1 or on the top (FIG. 5C) and bottom (FIG. 5D) of a membrane in platform 2.

### DETAILED DESCRIPTION

### I. Terms

Unless otherwise noted, technical terms are used according to conventional usage. Definitions of common terms in molecular biology may be found in Krebs et al., Lewin's Genes XI, published by Jones and Bartlett Learning, 2012 (ISBN 1449659853); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 2011 (ISBN 8126531789); and George P. Rédei, Encyclopedic Dictionary of Genetics, Genomics, and Proteomics, 2nd Edition, 2003 (ISBN: 0-471-26821-6). Definitions of common terms in radiation physics and radiotherapy may be found in Walter & Miller's Textbook of Radiotherapy: Radiation Physics, Therapy and Oncology, 7^{th} Edition, Churchill Livingstone, 2012 (ISBN 978-0443074868) and Radioactivity Radionuclides Radiation, Springer, 2005 (ISBN 978-3540211167).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of the disclosed technology, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the invention, the following explanations of specific terms are provided:

Cell Culture: Growth or maintenance of a population of cells in a defined set of conditions (such as culture medium, extracellular matrix, temperature, and/or time of culture) in vitro. In some examples, a cell culture includes a substantially pure culture (for example, substantially a single cell type). In additional examples a cell culture includes a mixed culture, such as co-culture of two or more types of cells. In further examples, a cell culture includes cells grown in contact with an extracellular matrix (such as one or more extracellular matrix components).

Membrane: A thin, substantially flat material or matrix that includes one or more pores. In some examples, a membrane is suitable for culturing cells on one or both sides. The membranes described herein can be coated with one or more extracellular matrix proteins or other molecules that facilitate cell attachment and or growth (such as collagen, laminin, fibronectin, L-ornithine, or a combination of two or more thereof). In some examples, the membrane is made of polyester or polycarbonate.

Radiation source: A structure (such as a wire, disk, or other form) that includes or is made of a radioactive material. The radiation source includes a radioactive material that emits gamma radiation, beta radiation, alpha radiation, or any combination thereof. Exemplary radioactive materials include ¹⁹⁸Au, ¹³⁷Cs, ^{137m}Ba, ⁶⁰Co, ¹⁹²Ir, ¹³¹I, ²¹³Bi, ¹⁰³Pd, ⁹⁰Sr, ¹⁰⁶Ru, or ²²⁶Ra.

Sample: Any material, including biological or non-biological materials. In some examples, a sample is a biological material, such as cells (including, but not limited to in vitro cultured cells), tissue (such as an organ or portion thereof), plant material, algae, or bacteria. In other examples, a sample is a non-biological sample, such as soil, rock, synthetic materials (such as polymers), or other human-made items. In some examples, a sample includes both biological and non-biological materials.

### II. Sample Platform Embodiments

Disclosed herein are sample platforms, which in some embodiments can be used for irradiation of a sample, such as a biological sample (for example, one or more types of cells). In other embodiments, the sample platforms can be used for culture of one or more types of cells (for example, co-culture of two or more types of cells). Platforms of the disclosure include a support structure including at least one well for holding a sample. The support structure can be any size or shape (for example, when viewed from the top having a square, rectangular, round, oval, triangular, or trapezoidal shape) sufficient to include at least one well (such as 1, 2, 3, 4, 5, 6, 12, 24, 48, 96, or more wells, for example, 1-100, 2-50, 10-40, or 30-60 wells) for holding one or more samples. In some embodiments, the platform also includes a radiation source that can be adjustably moved relative to the sample. In other embodiments, the platform includes a radiation source at a fixed distance relative to the sample; however, the distance between the sample and the radiation source can be adjusted based on the distance of the radiation source-holding portion of the platform from the sample. In further embodiments, a radiation source (separate from the platform) can be placed on or near the platform. This allows a user to control the amount of radiation to which the sample is exposed, for example, by adjusting the distance between the radiation source and the sample and/or the time of exposure.

In some embodiments, the support structure is a unit (such as a single structure) including sides (such as four sides if the support structure is square or rectangular) and a bottom. In some examples, the support structure also has at least a partial top surface, in which a well (such as an opening) is situated. In other embodiments, the support structure is formed by a plurality of substrates stacked on top of one other (such as 1, 2, 3, 4, 5, or more substrates) of the desired shape (such as a square or rectangular) with one or more openings (such as a round opening, for example to form a well). The substrates are stacked on a bottom substrate, which in some examples may be a solid substrate (for example, a substrate without any openings). In these embodiments, a support structure including a well of the desired depth can be constructed by a user on demand, by stacking a number of substrates to obtain the desired depth. In some examples, the height of the support structure (or well depth) is selected in order to allow placement of a radiation source at a desired distance from a sample that is placed in the well. The substrates can also include additional features, such as one or more channels and/or inlets or outlets (for example, for fluid insertion or removal). The support structure can be formed by stacking substrates which all have the same configuration, or a combination of substrates with different configurations.

In some embodiments, methods of making the devices disclosed herein utilize a layer-by-layer fabrication process by which patterned layers of films or sheet materials are produced and coupled to provide the substrates of the devices. In some embodiments, the layer-by-layer fabrication process includes patterning a desired elements (such as openings, channels, ports, etc.) into a substrate using a laser (e.g., laser ablation). In some embodiments, the layer-by-layer fabrication process can be performed manually or it can be automated. The layer-by-layer fabrication process can be used to produce substrates comprising the same or different configurations that, when the substrates are stacked vertically, will combine to form an integrated sample platform. In some embodiments, the substrates can be physically combined by vertically stacking and then clamping the substrates together, or they can be stacked and adhered together, such as by using an adhesive and/or a lamination technique known to those of ordinary skill in the art.

In some embodiments, a sample is placed in a well (either on a sample support structure in the well, or directly in the well, for example on the bottom surface of the well) in the support structure and the platform is utilized to culture the cells and/or test the effect of radiation on one or more properties or characteristics of the sample. The sample support can be suspended in the well (for example, using a transwell membrane) or can be attached to the sides of the well and extend across the well. In some examples, the sample includes cells, for example, one or more types of cells are cultured in vitro in the well, and the platform is utilized to test the effect of radiation on the one or more types of cells. In some examples, the well for holding the sample includes a sample support (such as a membrane) upon which the sample is placed or cells are cultured. However, the sample can also be placed in the well without any support or cells can be cultured on the bottom of the well without a support.

In some embodiments, the support structure also includes one or more chambers fluidly connected to the well(s) and/or one or more inlets and/or outlets fluidly connected to the well(s) (for example, for introducing or removing fluid in the chamber). In some examples, an inlet or outlet is connected to the chamber by a channel in the base of the support structure, or is formed by one or more of the substrates making up the support structure.

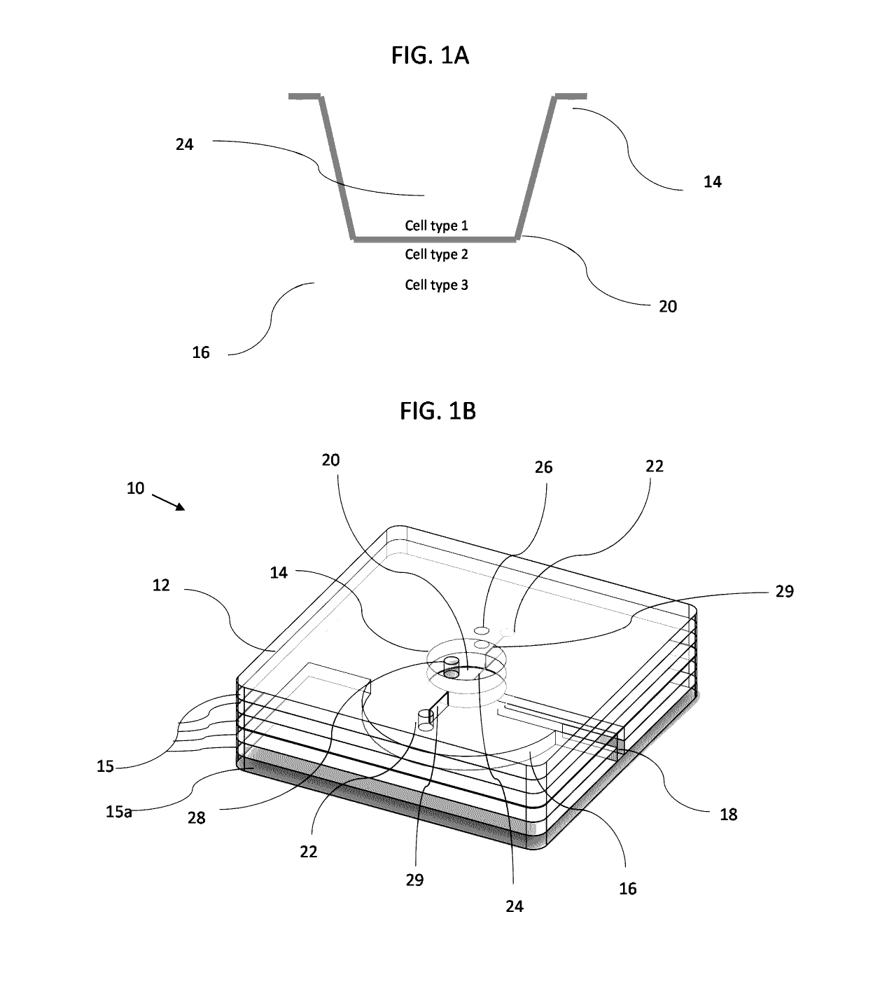

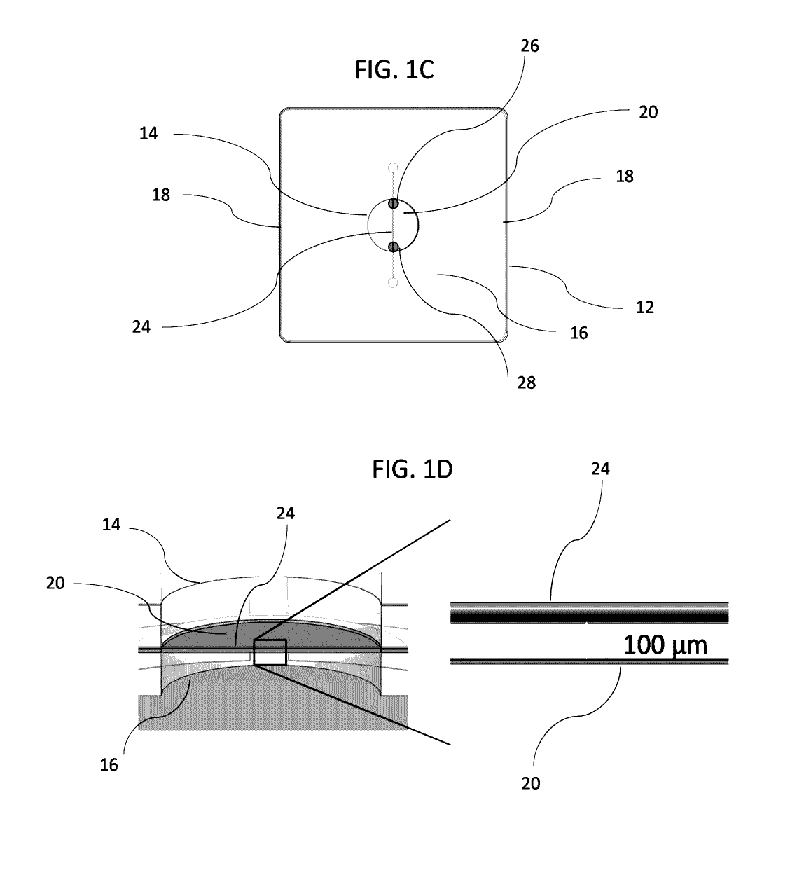

In some embodiments, the disclosed sample platforms are made of polymer material, including, but not limited to polyethylene terephtahalate glycol (PETG), polydimethylsiloxane (PDMS), polyester, polyethylene, polycarbonate, and/or acrylate. Each part of the platform (such as the support structure, plurality of substrates, sample cartridge, and/or sample support) can be made of the same or different material as each other. In embodiments including a sample support, the support can also be made of a polymer material. In particular examples, a sample support is a membrane compatible with in vitro cell culture, such as a polycarbonate, polyester, or polytetrafluoroethylene membrane. In some examples, the sample support is a Transwell® membrane (Corning Incorporated) or similar membrane. FIG. 1A illustrates in cross-section an exemplary well 14, showing a Transwell® membrane as the sample support 20 suspended over a chamber 16 with a radiation source 24 passing over sample support 20.

Referring now to FIG. 1B, sample platform 10 is shown in perspective view, according to one embodiment (“platform 1”). Sample platform 10 includes a support structure 12 including a well 14 for holding a sample. The support structure 12 can be of any convenient size to accommodate a well 14 of the desired diameter. In some examples, the well 14 has a diameter of about 1 mm to about 100 mm (such as about 4 mm to about 75 mm, about 6 mm to about 50 mm, about 4 mm to about 24 mm, or about 10 mm to about 50 mm). In one non-limiting example, the well 14 has a diameter of about 6.5 mm. In some embodiments, support structure 12 is made of a plurality of substrates 15 stacked on top of one other. In some examples, the bottom substrate 15ais solid (e.g., does not include any openings).

The support structure 12 also includes a chamber 16 below and in fluid communication with the well 14 and at least one port or channel 18 for adding and/or removing fluids fluidly connected to the chamber. The sample platform 10 of this embodiment includes a sample support 20 that is inserted in the well 14 and is suspended above the chamber 16. In some examples, sample support 20 is a Transwell® membrane. In some embodiments, the support structure includes additional inlet and/or outlet ports 26 and 28 that are used for introducing and/or removing cell culture medium, medium supplements, or other treatments for the cells.

Radiation source supports 22 are on opposite sides of the well 14 with a radiation source 24 extending therebetween. The radiation source 24 is inserted in slots 29 connected to radiation source supports 22 on each side of well 14, and in some embodiments, the distance from the sample support 20 can be adjusted by moving the radiation source 24 vertically in the slots 29. In particular embodiments, the radiation source 24 is a wire, such as a wire plated with a radioactive material (for example ¹⁹⁸Au).

As illustrated in FIG. 1C, radiation source 24 (e.g., a wire) passes through well 14 above the sample support 20. Well 14 is in support structure 12, which includes chamber 16 and ports 18, 26, 28 for adding and/or removing fluids in the chamber. FIG. 1D is a schematic cross-section of an exemplary well portion of a platform (left), with a higher magnification view of the radiation source 24 (wire) suspended above the sample support 20, by 100 μm in this embodiment (right).

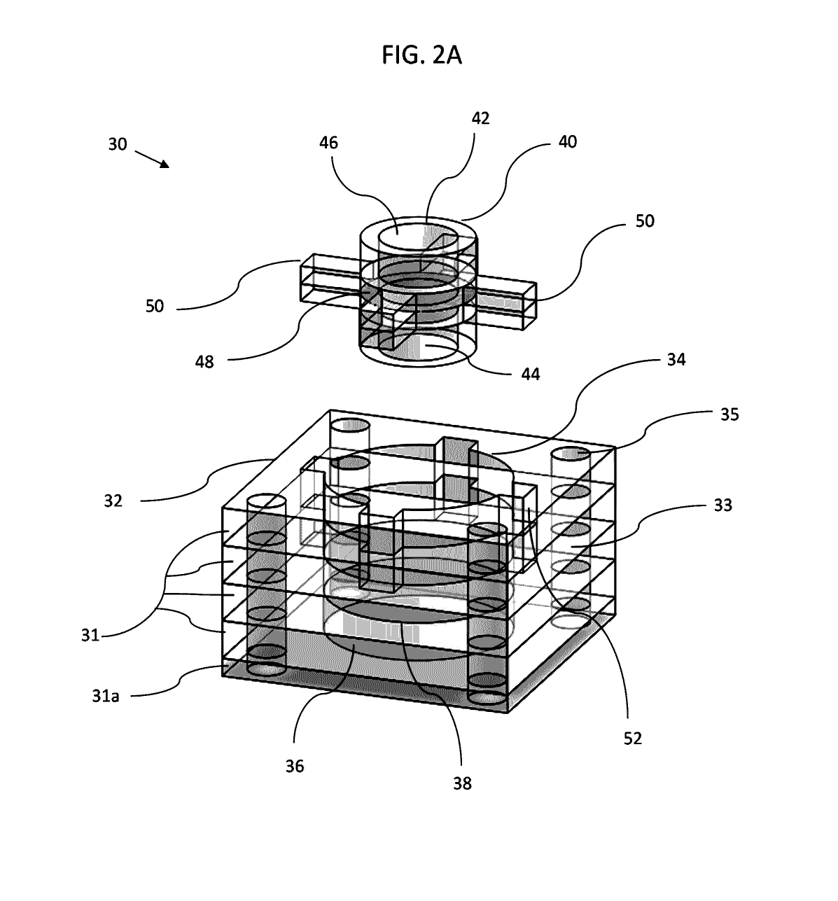

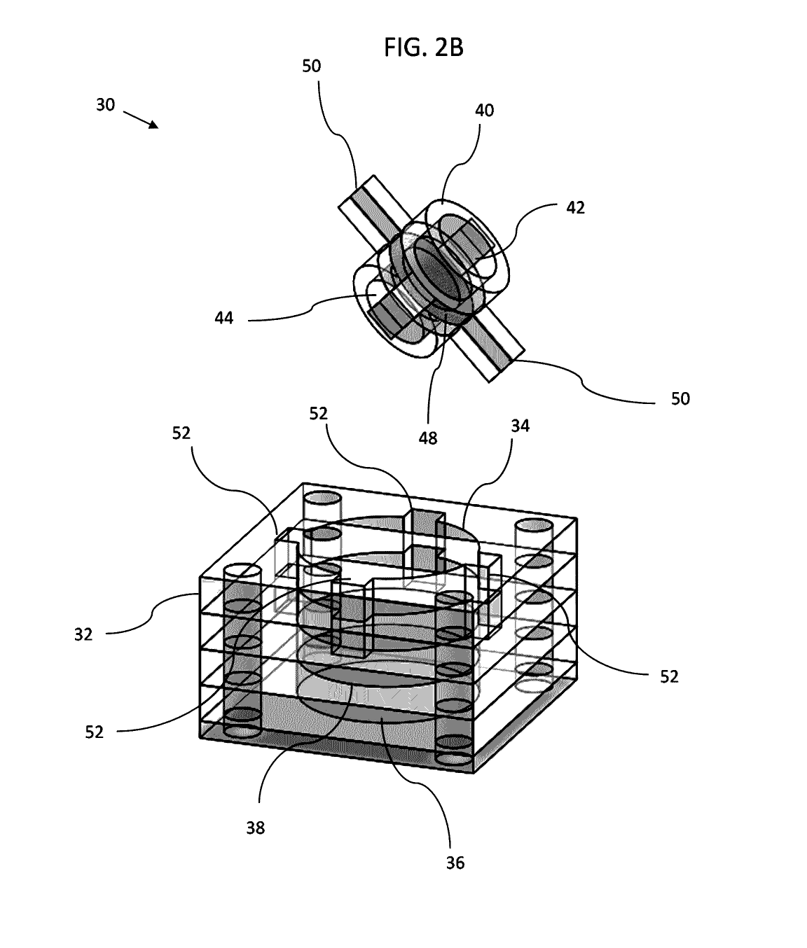

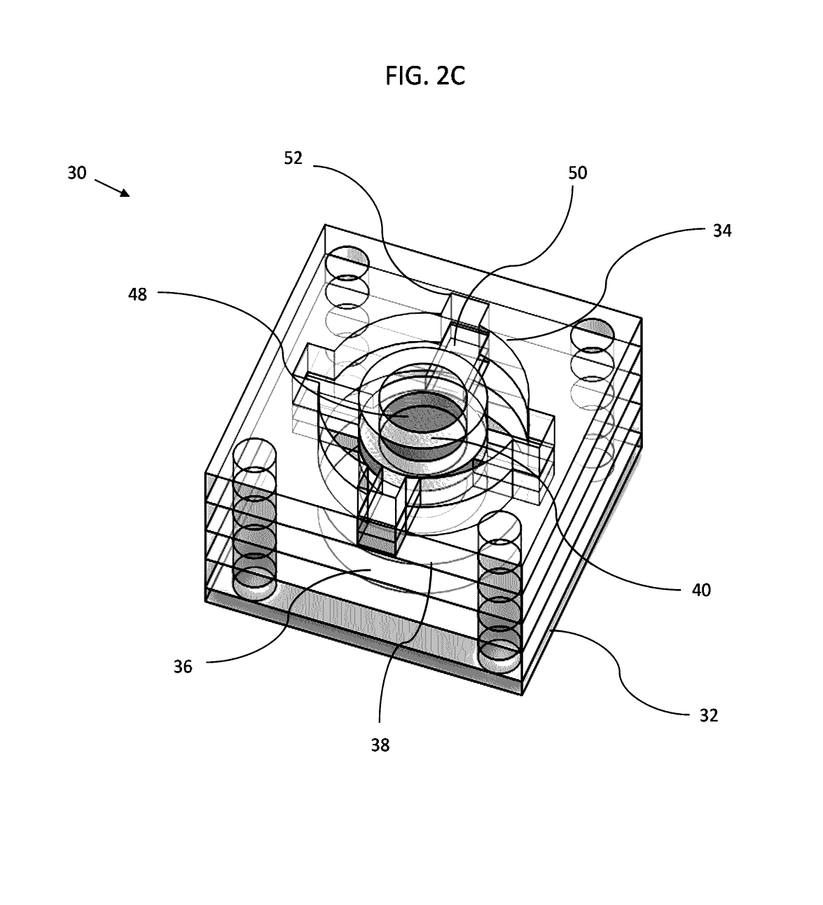

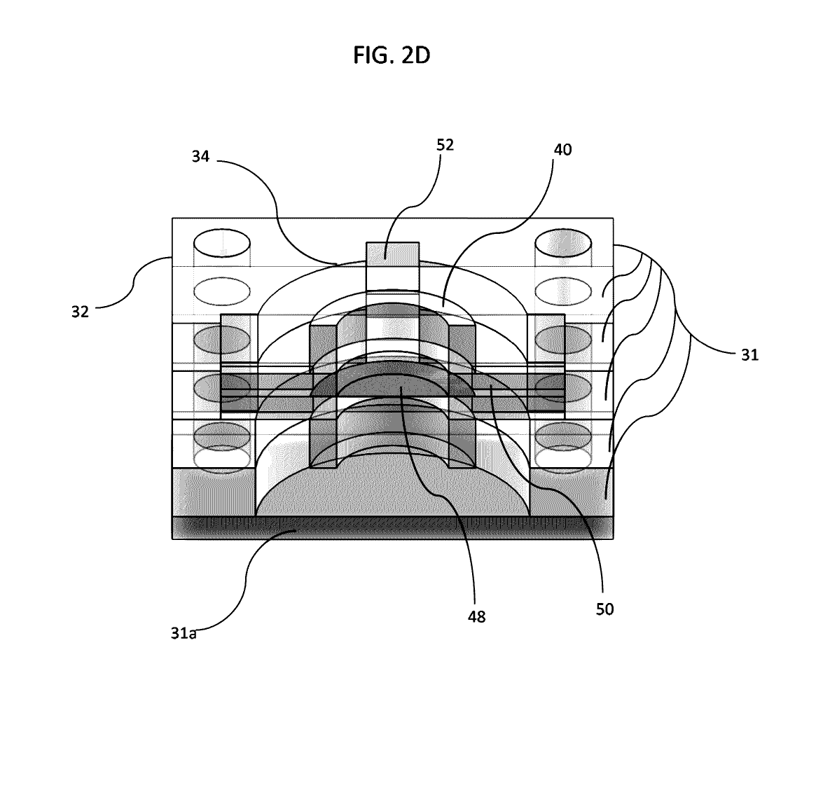

Referring now to FIGS. 2A-2B, sample platform 30 is shown in perspective views, according to another embodiment (“platform 2”). Sample platform 30 includes a support structure 32 including a well 34. In this embodiment, the support structure is formed from stacked substrates 31, placed on a bottom substrate 31a. The substrates are aligned using a pin or post 33 that inserts into alignment holes 35 on each of the substrates 31. The support structure 32 also includes a chamber 36 below and in fluid communication with the well 34. Sample platform 30 includes sample cartridge 40 including opposite top and bottom openings 42, 44 and an elongated bore 46 extending therethrough. Sample cartridge 40 of this embodiment includes a sample support 48 inserted in the elongated bore 46. The sample support has a top surface and a bottom surface. In some embodiments, sample support 48 is a disk or membrane inserted in sample cartridge 40. In some examples, the sample support is a Transwell® membrane (Corning Incorporated) or similar membrane. In some examples, sample cartridge 40 has an interior diameter of about 1 mm to about 100 mm (such as about 4 mm to about 75 mm, about 6 mm to about 50 mm, about 4 mm to about 24 mm, or about 10 mm to about 50 mm). In one non-limiting example, sample cartridge 40 has an interior diameter of about 6.5 mm. In further examples, the support structure 32 includes at least one inlet or outlet fluidly connected to well 34 and/or chamber 36 (not shown). The at least one inlet or outlet can be used to introduce or remove fluid from the well or chamber (for example, when the sample cartridge is placed in the support structure, as discussed below).

The sample cartridge 40 is insertable in support structure 32. Thus, well 34 in support structure 32 is of a diameter that can accommodate the outer diameter of sample cartridge 40. Sample cartridge 40 includes two or more (such as 2, 3, 4, or more) extensions 50 that are insertable into corresponding slots 52 on the support structure 32 when aligned. As illustrated in FIG. 2B, in some embodiments, sample cartridge 40 can be inserted in well 34 in at least two orientations, for example with the bottom surface of sample support 48 facing toward chamber 36 or with the top surface of sample support 48 facing toward chamber 36 (e.g., inverting the sample cartridge).

Referring now to FIGS. 2C and 2D, sample platform 30 is shown with sample cartridge 40 inserted in well 34 (FIG. 2C). As shown in a cross-sectional view (FIG. 2D), when inserted in support structure 32, sample cartridge 40 is held in place by sample cartridge extensions 50 being held in slots 52 in substrates 31, which do not extend into support structure 32 to the bottom substrate 31a. In this manner, sample support 48 is suspended in well 34.

### III. Methods of Use

Disclosed herein are methods of using the sample platforms described herein. In some embodiments, the sample platforms are used to grow a sample (such as a sample including one or more types of cells). In some embodiments, the disclosed platforms are used to expose samples (including, but not limited to one or more cell types) to radiation, for example, controlled amounts of radiation. Thus, representative methods provided herein can be used to determine the effects of radiation on one or more properties of the sample. In other embodiments, the disclosed platforms are used for culturing one or more cell types, without exposing the cells to any radiation. In these embodiments, the radiation source is not included in, or placed on or near, the platform.

In some examples, the sample is a biological sample, and the methods are used to evaluate the effect of radiation on properties such as cell growth, cell morphology, gene expression (for example, presence or amount of one or more RNAs and/or proteins), cellular signaling (for example, secretion of one or more signaling molecules), or cellular contact. One of ordinary skill in the art can identify other properties that can be evaluated using the methods disclosed herein.

In some embodiments, a sample is placed in the well of the platform, for example on the chamber or on a sample support suspended in the well. In particular embodiments, the methods include culturing cells on the chamber and/or sample support in the well. In some examples, the sample support is a membrane (such as a Transwell® membrane) and cells can be cultured on one or both sides of the sample support. In some examples, the methods include culturing one type of cells on one or more of the chamber and the sample support. In other examples, the methods include culturing two or more types of cells on one or more of the chamber and sample support. For example, one type of cells is cultured on the top side of the membrane and a different type of cells is cultured on the bottom side of the membrane. In other examples, one type of cell is cultured on one or both sides of the membrane and a different type of cell is cultured on the chamber. In a still further example, one type of cell is cultured on the top side of the membrane, a second type of cell is cultured on the bottom side of the membrane, and a third type of cell is cultured on the chamber. One of ordinary skill in the art can select the number and type of cells to be cultured in the platform, depending on the system being studied.

In some embodiments (for example, platform 2), cells are cultured on a sample support included in the sample cartridge. For example, the sample cartridge is inserted in the support structure and cells and culture medium are placed on the upper (top) side of the support. Culture medium can also be placed in the well (or chamber) under the sample cartridge, such that the lower (bottom) side of the sample support is also contacted with the medium. The medium in contact with the upper and lower sides of the sample support can be the same medium, or different media. After a period of time (such as a period of time sufficient for the cells on the upper side of the membrane to attach to the membrane, and/or for a period of time sufficient to obtain the desired number of cells or particular cell characteristics), the sample cartridge is removed from the support structure, inverted, and reinserted into the support structure. Thus, the previously lower side of the sample support becomes the upper side and the previously upper side of the sample support (now including the cells) becomes the lower side. Both the new upper side of the sample support is contacted with cells and culture medium and the new lower side of the sample support is also contacted with culture medium (in the well of the support structure). The medium in contact with the upper and lower sides of the sample support can be the same medium, or different media. The cells can be treated in different ways (for example, exposed to one or more test compounds) or simply cultured in standard cell culture medium. Following culture and/or treatment, one or more properties of the cells (such as cell proliferation, cell death, gene expression, cell signaling, and/or cell contacts) is evaluated.

In one non-limiting example, the methods include culturing a myoblast cell line (such as C2C12 cells) on one or both sides of a membrane sample support. In another non-limiting example, the methods include culturing endothelial cells on one side of a membrane sample support and culturing neuronal cells or fibroblast cells on the other side of the membrane sample support. In a further non-limiting example, myoblast cells (such as C2C12 cells) are cultured on one side of a membrane sample support and neuronal cells (such as NSC-34 motor neuron cells) are cultured on the other side of the membrane sample support under conditions that support differentiation of the cells and formation of neuromuscular junctions (NMJs) or NMJ-like structures. In this example, a membrane with pores of a diameter sufficient to allow neurites to pass through to connect to the neuronal cells, while retaining the cells on the surface of the membrane (e.g., pore diameter of about 1-5 μm). One of ordinary skill in the art can select cell type(s) for culturing in the devices disclosed herein.

In some embodiments, after placing the sample in the well or sample cartridge (or after a sufficient time for cell cultures to become established), the sample is irradiated, for example, by placing a radiation source at a selected distance from the sample. The radiation source is placed at a distance that provides the desired radiation intensity. In some embodiments, the radiation source is a wire that extends through the well and is suspended above the sample, for example, by inserting a radiation source in the radiation support of platform 1. In other embodiments, the radiation source is a sheet or disk including radioactive material that is placed above or below the well of the platform (such as platform 2). In some examples, the distance of the radiation source from the sample support is controlled by the height of the support structure or the number of substrates in the support structure. One of ordinary skill in the art can adapt the disclosed platforms to include other means for adjusting the distance between the radiation source and the sample support.

The radiation source is placed in or on (or near) the support structure at the selected distance from the sample (for example, about 10 μm to 1 mm (such as about 10-100 μm, about 50-250 μm, about 100-500 μm, about 250-1000 μm, or about 10 μm, about 20 μm, about 50 μm, about 75 μm, about 100 μm, about 250 μm, about 500 μm, about 750 μm, or about 1000 μm) from the sample. After a period of time, such as about 0.1 seconds to about 2 hours (such as about 1 second to 1 minute, about 10 seconds to 10 minutes, about 1 minute to about 30 minutes, about 5 minutes to about 1 hour, about 20 minutes to about 90 minutes, or about 1 hour to about 2 hours), the radiation source is removed from the support structure. If the sample includes cells, they can optionally be maintained in culture following radiation exposure to evaluate the effect of the radiation on one or more properties (such as cell proliferation, cell death, gene expression, cell signaling, and/or cell contacts) or can be processed immediately to analyze one or more properties (including, but not limited to, gene expression, cell number, cell proliferation, cell death, morphology, and so on).

### EXAMPLES

The following examples are illustrative of disclosed embodiments. In light of this disclosure, those of skill in the art will recognize that variations of these examples and other examples of the disclosed technology would be possible without undue experimentation.

### Example 1

### Cell Culture on Membranes in Platform 1

This example describes culture of different cell types on membranes in standard cell culture dishes and in a platform as described herein.

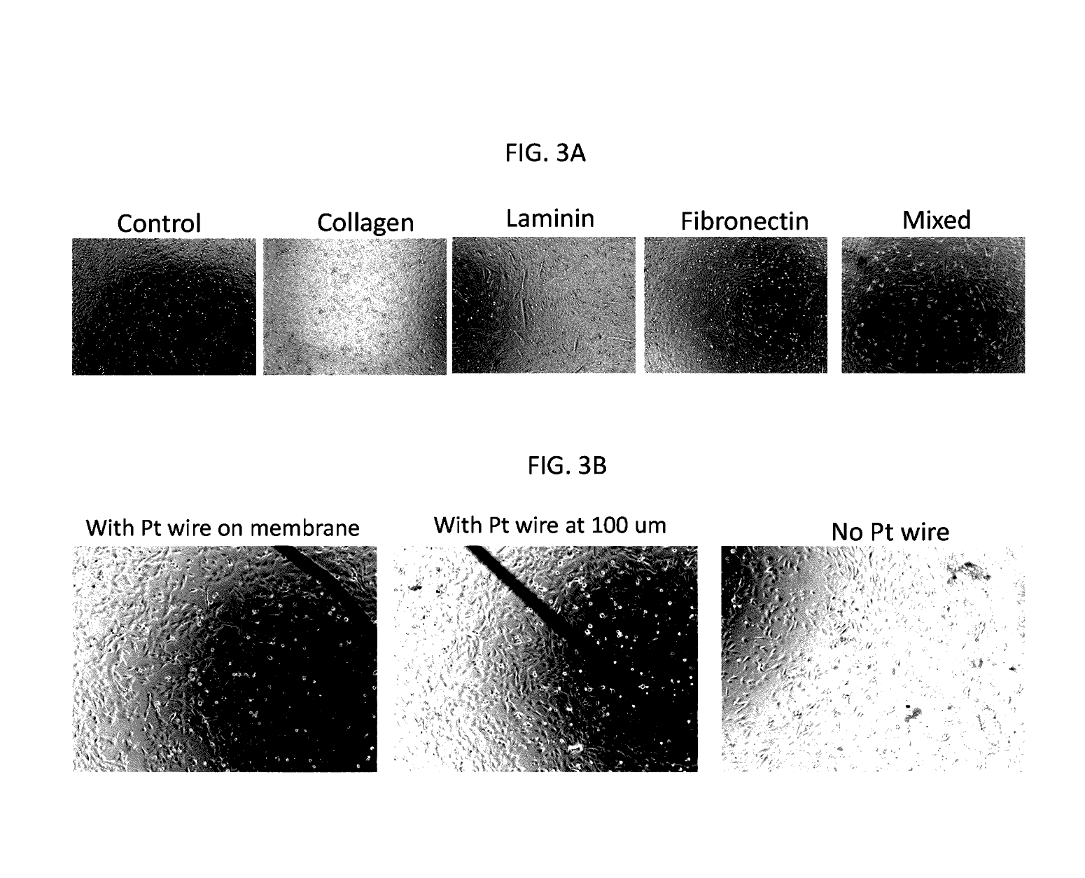

C2C12 myoblast cells were cultured in 12 well culture plates on Transwell® membranes (Corning Incorporated, Tewksbury, Mass.) or Transwell® membranes coated with collagen, laminin, fibronectin, or a mixture (FIG. 3A). The cells were also grown on Transwell® membranes (polyester membrane with 0.4 μm pores coated with collagen, laminin, fibronectin, or 1:1:1 mixture) in a well in a device with the platform 1 design. Cells were cultured on the membrane for 3 days in DMEM with 10% FBS and 1% penicillin/streptomycin. Cells were grown with platinum wire placed directly on the membrane, a platinum wire suspended 100 μm above the membrane, or without a platinum wire (FIG. 3B).

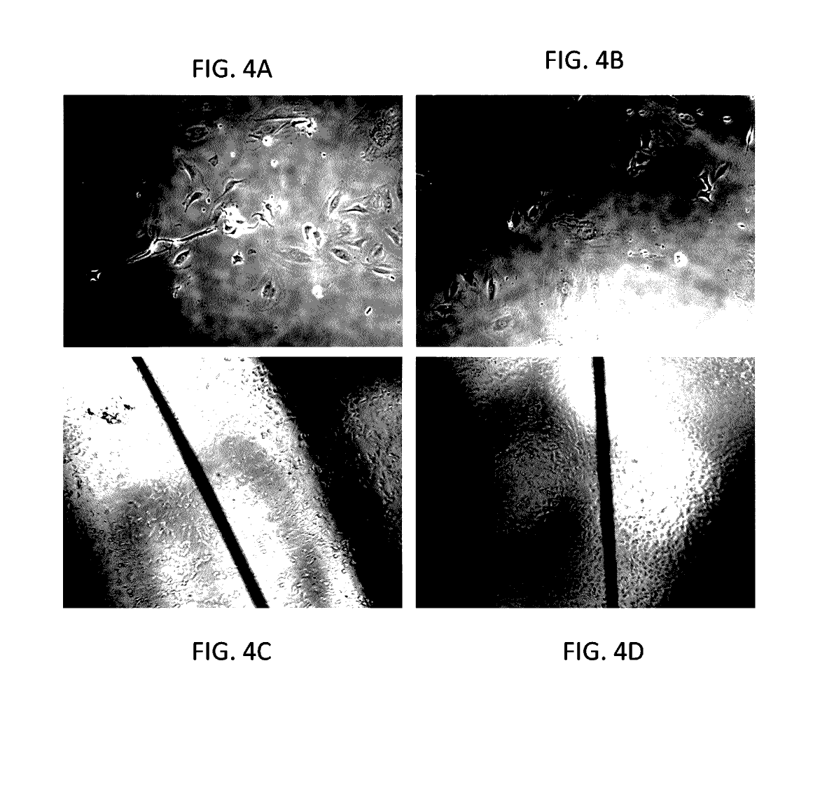

Endothelial cells and neuronal cells were cultured on opposite sides of a Transwell® membrane. Mouse motor neuron cells (NSC-34) were maintained in DMEM contained 10% FBS with 1% penicillin/streptomycin. Human microvascular cells were cultured in microvascular endothelial cell growth medium containing 1% penicillin/streptomycin. The membrane and cells were in a platform of design 1, with a platinum wire suspended above the cells (FIGS. 4A-4D). FIG. 4A shows the neuron cells can grow on the bottom side of membrane. FIG. 4B shows that after 3 days neuron cells still survive in the device. FIG. 4C shows endothelial cells (human microvascular cells) can grow on the top side of membrane and the wire was set above the membrane about 100 μm. FIG. 4D shows the endothelial cells still survived after 3 days of cell culture in the device.

### Example 2

### Myoblast Cell Culture in Platform 2

This example describes culture of myoblast cells on membranes in an irradiation platform.

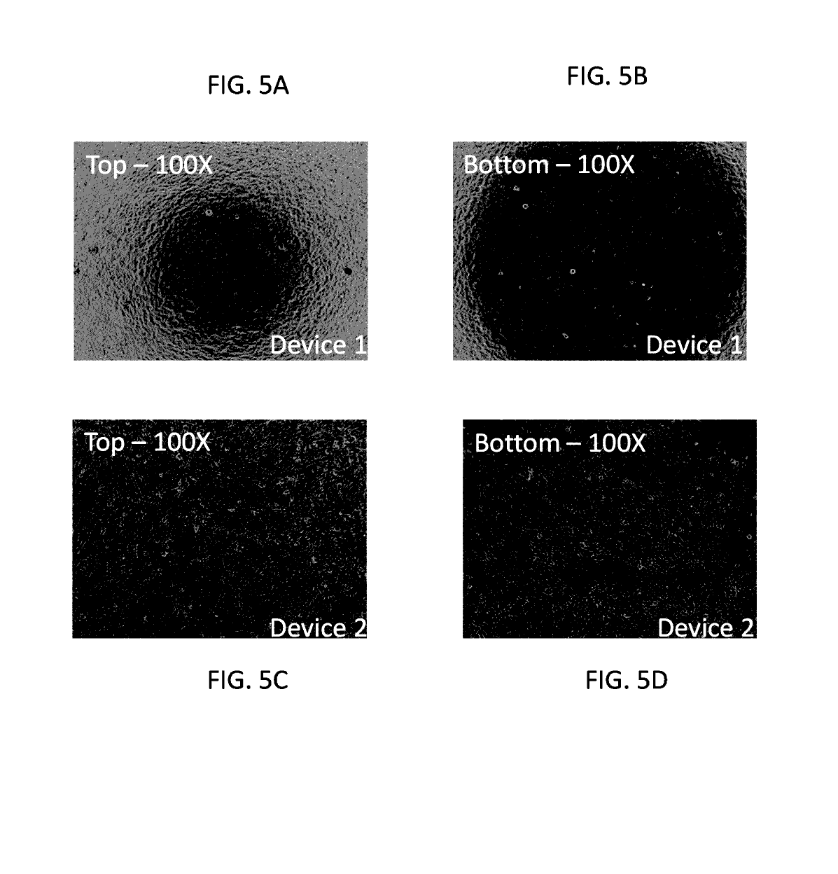

C2C12 cells were cultured as described in Example 1, except they were plated and grown on both sides of the Transwell® membrane. The membrane was present in either platform 1 or platform 2 (FIGS. 5A-5D). FIGS. 5A-5B show growth of C2C12 cells on both sides of the membrane in device 1. FIGS. 5C-5D show growth of C2C12 cells on both sides of membrane in device 2.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A sample platform comprising:
a support structure comprising a plurality of stacked substrates forming at least one well and comprising one or more fluid inlets and/or outlets fluidly connected to the at least one well; and
a removable sample cartridge comprising a sample support, wherein the sample cartridge can be inserted in or removed from the well of the support structure.

2. The platform of claim 1, wherein the sample support comprises a membrane.

3. The platform of claim 2, wherein the membrane comprises polyester, polycarbonate, or polytetrafluoroethylene.

4. The platform of claim 1, wherein the support structure further comprises one or more slots for insertably receiving the sample cartridge.

5. The platform of claim 1, wherein the support structure and/or the sample cartridge is polycarbonate, acrylate, polyethylene terephtahalate glycol, or polydimethylsiloxane.

6. The platform of claim 1, wherein the sample cartridge further comprises one or more extensions, wherein the one or more extensions are insertable in the one or more slots on the support structure.

7. A sample platform assembly comprising:
a support structure comprising plurality of stacked substrates forming at least one well and one or more fluid inlets and/or outlets fluidly connected to the at least one well; and
a removable sample cartridge comprising a sample support and a sample on the sample support, wherein the sample cartridge is inserted in the well of the support structure.

8. The assembly of claim 7, wherein the sample support comprises a membrane.

9. The assembly of claim 8, wherein the membrane comprises polyester, polycarbonate, or polytetrafluoroethylene.

10. The assembly of claim 7, wherein the support structure and/or the sample cartridge is polycarbonate, acrylate, polyethylene terephtahalate glycol, or polydimethylsiloxane.

11. The assembly of claim 7, wherein the support structure further comprises one or more slots for insertably receiving the sample cartridge.

12. The assembly of claim 7, further comprising a radiation source placed on or near the support structure.

13. The assembly of claim 12, wherein the radiation source is a sheet, disk, or wire comprising radioactive material.

14. The assembly of claim 7, wherein the sample cartridge further comprises one or more extensions, wherein the one or more extensions are insertable in the one or more slots on the support structure.

15. A method, comprising:
placing a sample on the sample support in the sample cartridge of the sample platform of claim 1;
inserting the sample cartridge in the well of the support structure;
incubating the sample for a selected period of time;
removing the sample cartridge; and
evaluating one or more characteristics of the sample.

16. The method of claim 15, wherein the sample is a biological sample.

17. The method of claim 16, wherein the sample comprises one or more cells or one or more cell types.

18. The method of claim 15, wherein the sample support comprises a membrane having two sides.

19. The method of claim 18, wherein cells are cultured on one or both sides of the membrane.

20. The method of claim 19, wherein evaluating one or more characteristics of the sample comprises measuring one or more of cell proliferation, cell death, gene expression, cell signaling, and cell contact.

21. The method of claim 15, further comprising irradiating the sample prior to removing the sample cartridge.
